(19) United States
(12) Patent Application Publication
(10) Pub. No.: US 2009/0312407 A1
(43) Pub. Date: Dec. 17, 2009

(54) Synthetic Flavonoids and Pharmaceutical Compositions and Therapeutic Methods of Treatment of Cancer and other Pathologies

(76) Inventors: Kinfe Redda, Tallahassee, FL (US); Chavonda Janeebra Mills, Decatur, GA (US); Nelly Mateeva, Tallahassee, FL (US)

Correspondence Address: Dennis Clarke, 7422 Old Maple Square, McLean, VA, 22102, US

(21) Appl. No.: 11/957,462

(22) Filed: Dec. 16, 2007

(51) Int. Cl. A61K 31/352; C07D 311/26; C07C 49/84; A61K 31/12; A61P 35/00

(52) U.S. Cl. 514/456; 549/403; 568/334; 514/685

(57) ABSTRACT

A compound, pharmaceutical composition and method for the treatment of mammals wherein the active therapeutic agent is a compound having the structure:

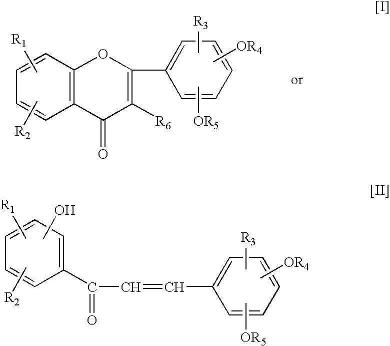

wherein:
   R₁ is an electronegative substituent,
   R₂ is R₁ or alkyl,
   R₃ is H or O-alkyl,
   R₄ and R₅ are the same or different and are alkyl and
   R₆ is H or OH.

### OTHER APPLICATIONS

This application claims the benefit of provisional patent application, Ser. No. 60/872,624, filed, Dec. 4, 2006; the entire contents and disclosure of which is incorporated herein by reference.

### FIELD OF THE INVENTION

The present invention relates to novel therapeutic agents suitable for the treatment of mammals, in particular, those afflicted with cancer.

### BACKGROUND OF THE INVENTION

The entire disclosures and contents of each reference and patent referred to herein is incorporated by reference.

Flavonoids (benzo-y-pyrones), as shown in Scheme 1 are a class of about 4,000 natural compounds present in all vascular plants. They appear to exhibit a wide range of biological activities including anti-oxidative [H. Bao et al, Food Chemistry, 86, 517 (2004); T. Miura et al, Food and Chemical Toxicology 41, 759 (2003)], anti-inflammatory [J. J. A. Hendricks et al, Journal of Experimental Medicine, 200, 1667 (2004); H. Y. Lin et al, Journal of Cellular Physiology, 202. 579 (2005); Y. S. Chi et al, Biochem. Pharmacol, 62, 1185 (2001)], cancer suppressing [T. Zhang et al, Bioorganic & Medicinal Chemistry, 12, 6097 (2004); C. A. Rowe et al, Journal of Medicinal Food, 7, 402 (2004). X. Zheng et al, Bioorganic and Medicinal Chemistry Letters, 13, 3423 (2003; X. Zheng et al, Bioorganic and Medicinal Chemistry Letters, 13, 881 (2003] and antiviral (anti-HIV) [W. Wang et al, Antiviral Research, 64, 189 (2004); T. B. Ng, et al, Life Sci., 61, 933 (1997); A. J. Vlietnick et al, Plant Flavonoids in Biol. And Medicine II; Biochem. Cell. and Medicinal Properties, 283 (1988).]. The average human diet contains about 1 g of flavonoids per day, assimilated through fruits, vegetables, red wine, tea etc. [G. Di Carlo et al, Life Sciences, 65, 337 (1999)].

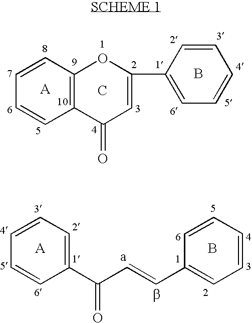

A family of flavonoids are known to be selectively toxic to multidrug resistant cancer cells. Several recent studies have linked the regulation of P-glycoprotein (Pgp) gene expression to the expression of the drug-metabolizing P450 genes, with the speculation that, in normal cells, P-glycoprotein may function in conjunction with the P450 enzymes in the detoxication of xenobiotics. Indeed, benzo[a]pyrene, an inducer and a substrate for cytochrome P450Ia1, is also a substrate for P-glycoprotein. While investigating the coordinated regulation of these genes following exposure of multidrug resistant (MDR) cells to inducers of P450 gene expression, it was observed that one of these inducers, β-naphthoflavone (βNF), was considerably more toxic to multidrug resistant cells than to their drug-sensitive counterparts. This collateral sensitivity to βNF and other flavonoid compounds has now been further investigated in several multidrug resistant cell lines. For a further discussion of the anti-cancer properties of flavonoids, see US. H1,427; U.S. Pat. Nos. 5,336,685; 6,706,865 and 6,528,042.

Most of the flavonoids have been isolated from plants in order to test their biological properties; others have been synthetically produced in search of more potent drug candidates.

The structure-activity relationship (SAR) in flavonoids is empirical, i.e., based on numerous data from testing different compounds. Although there are a lot of data in the literature, it is very difficult to outline a clear tendency that will lead to an optimized structure of high activity and low toxicity.

Among the most studied compounds, quercetin and baicalin are polyhydroxylated flavones [L. M. Larocca et al, J. Ural., 152, 1029 (1994) 1417; K. Ono et al, Biochem. Biophys. Res. Commun., 160, 982 (1989); B. Q. Li et al, Cell And Mol. Biol. Res., 39, 119 (1993)]. It is believed that the important structural features leading to high biological activities are the presence of 5-OH and 3-OH groups. The latter, however, is considered to be responsible for some mutagenic properties observed in quercetin, which disappeared after this group was methylated. Their solubilities can be modified through selective introduction of lipophilic and hydrophilic substituents. The most popular approach is to balance the number of the alkyl substituents and free hydroxyl groups.

The incorporation of electronegative elements, such as halogens and nitro groups in the flavonoid structure, introduce new patterns of biological properties. 4′,6-dichloroflavan has been reported to be a potent antirhinovirus compound. Halogenated and nitro-substituted flavones exhibit structure-dependent aryl hydrocarbon receptor (AhR) agonist and antagonist activities comparable to that observed for 2,3,7,8-tetrachlorodibenzo-p-dioxin (TCDD) [Y. F. Lu et al, Biochemical Pharmacology. 51. 1077 (1996)]. 8-Iodo. 8-bromo and 8-trifluoromethyl derivatives of chrysin exhibit strong activities against human gastric adenocarcinoma cell lines (SGC-7901) and colorectal adenocarcinoma (HT-29) cells. One of the most recent studies reports the effect of some flavonoids on the central nervous system.

Halogenated flavanones and flavones are considered potential benzodiazepine receptor ligands. Indeed, 6-bromoflavone and 6-bromo-3′-nitroflavone showed activities close or higher than that of diazepam [P. Bovichelli et al, Tetrahedron Letters, 43, 5563 (2002)].

It is an object of the present invention to provide novel chalcones and flavones substituted with electronegative groups useful for the treatment of mammals (man or animal), in particular, mammals afflicted with cancer and methods for their synthesis.

### SUMMARY OF THE INVENTION

The above and other objects are realized by the present invention, one embodiment of which relates to novel compounds having the structure:

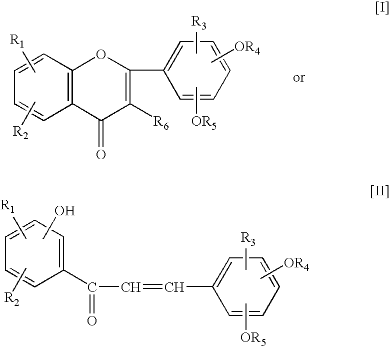

wherein:
R₁ is an electronegative substituent,
R₂ is R₁ or alkyl,
R₃ is H or O-alkyl,
R₄ and R₅ are the same or different and are alkyl, and
R₆ is H or OH.

A further embodiment of the invention concerns methods for the syntheses of the above described compounds comprising synthesizing the chalcones by the Claisen condensation of appropriately substituted acetophenones and benzaldehydes, preferably in ethanol and synthesizing the flavones by a three step Baker-Venkataraman rearrangement.

An additional embodiment are pharmaceutical compositions comprising therapeutically effective amounts for the treatment of cancer of a compound or mixture of compounds as described above and pharmaceutically acceptable carriers or excipients therefore.

A further embodiment of the invention comprises methods for the treatment of cancer in mammals in need thereof comprising administering thereto a therapeutically effective amount of a compound or mixture of compounds as described above.

Still further embodiments of the invention relate to (1) articles comprising packaging material which contains at least one biologically active agent contained within the packaging material, wherein the at least one therapeutic agent is effective for the treatment of a subject requiring treatment for cancer, and wherein the packaging material comprises a label which indicates that the therapeutic agent can be used for at least ameliorating the symptoms with which the subject is afflicted, and wherein the at least one biologically active agent is a compound or mixture of compounds as described above and (2) kits comprising, in separate packages: (A) a therapeutically effective amount of a compound or mixture of compounds as described above and (B) a carrier or excipient therefore or a biologically active agent different from the compound or mixture of compounds.

### BRIEF DESCRIPTION OF THE DRAWINGS

FIGS. 1-3 are graphical depictions of the pharmacological properties of the compounds of the invention.

FIG. 4 is a tabular representation of the compounds of the invention and their structures.

### DETAILED DESCRIPTION OF THE INVENTION

The present invention is predicated on the discovery that chalcones and flavones substituted as shown above have unexpected and unobvious therapeutic activities, in particular, anti-cancer activities.

The chalcone derivatives (5-10 in scheme 1 below and Table 1) were synthesized by Claisen condensation of appropriately substituted acetophenones and benzaldehydes, preferably in ethanol in the presence of 50% aqueous KOH. The reaction times as well as the yields vary depending upon the corresponding reagents. The compounds containing the dimethoxybenzaldehyde unit were obtained with lower yields than the trimethoxy derivatives. The crude products were contaminated with some starting materials which could easily be removed by column chromatography on silica gel using CH₂Cl₂:hexane as an eluent (Scheme 3).

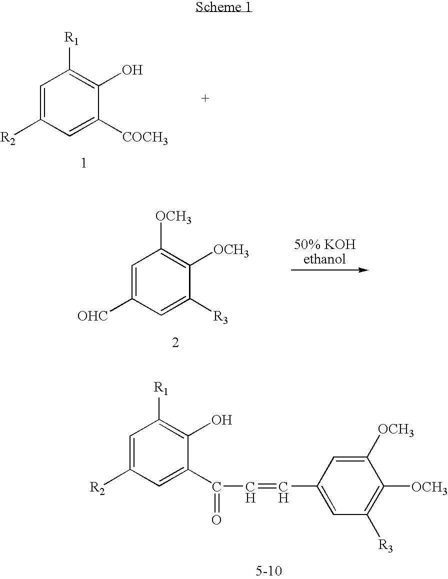

Flavones (17-22; Table 1) were synthesized using a three step Baker-Venkataraman rearrangement. The crude product was purified by recrystallization and Chromatotron chromatography using CH₂Cl₂:MeOH as an eluent (Scheme 2).

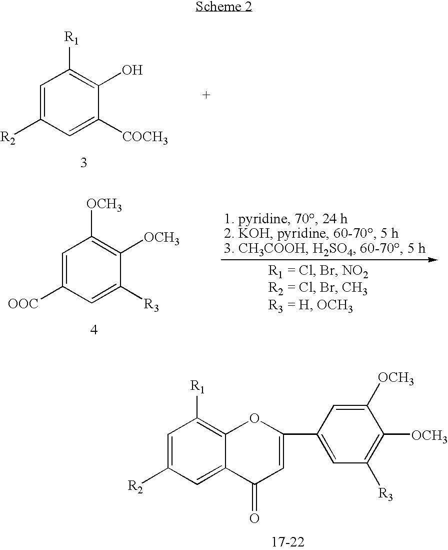

The structures of the synthesized compounds were confirmed by IH-NMR, IR and elemental analysis (table 2). The compounds were subjected to tests that confirmed their cyclooxygenase-1 and cyclooxygenase-2 binding and anti-HIV and anti-cancer activities.

In the structural formulas I and II above, the alkyl groups may be straight- or branch-chain and may contain from 1 to 6 carbon atoms. Preferred alkyl groups for R₂, R₃, R₄ and R₅ are methyl, ethyl, propyl, isopropyl and tertiary butyl; most preferred being methyl.

The electronegative substituents on the molecules depicted in formulas I and II may be halogen, preferably chloro and bromo, and nitro.

As used herein, “treating” describes the management and care of a patient (human or animal) for the purpose of combating a disease, condition, or disorder and includes the administration of a compound of the present invention to prevent the onset of the symptoms or complications, alleviating the symptoms or complications, or eliminating the disease, condition or disorder.

The dosage of compound employed will depend in each case upon the nature of the cancer being treated; on absorption, inactivation and excretion rates of the drug, as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions. Generally, it is preferred to utilize orally administered compositions such as tablets or capsules containing 50-100 mg, administered twice a day for an adult patient weighing 70 kg (154 lbs). It will be understood by those skilled in the art, however, that any suitable mode of administration and appropriate dosage may be employed in the practice of the invention.

Although the methods and compositions of the invention may be employed to treat any cancer sensitive to the compounds of the invention, the invention is particularly suited for the treatment of breast and prostate cancer. Breast cancer is the most prevalent cancer in women and second leading cause of cancer deaths among U.S. women. It has been estimated that there were 215,990 new breast cancer cases in the United States (accounting for 32% of all cancer cases involving women), and 40,110 breast cancer deaths (15% of all cancer deaths among women) in 2004. When looking at the black female population, the death rates were 30.6 and 29.7 per 100,000 for the 3-year period of 2000-03 and 2002-04, respectively. The American Prostate Cancer Foundation states that prostate cancer is the most common non-skin cancer in America. In 2006, over 232,000 men will be diagnosed with prostate cancer, and over 30,000 men will die from it. One new case occurs every 2.5 minutes and a man dies from prostate cancer every 17 minutes. After lung cancer, prostate cancer is the leading cause of cancer-related deaths among men in the U.S.A nonsmoking man is more likely to get prostate cancer than lung, bronchus, colon, rectal, bladder, lymphoma, melanoma, oral and kidney cancers combined.

In the examples below, melting points were determined on a Gallenkamp Melting Point Apparatus and were uncorrected. Infrared spectra were obtained on a Perkin Elmer FTIR 1430 spectrophotometer, using KBr pellets. ¹H nmr and ¹³C nmr spectra were obtained with a Brucker HX-300 spectrometer and the chemical shifts were reported as parts per million (5 ppm) downfield.

Column chromatography as well as Chromatotron 8924 (Harrison Research) instruments were used for purification of the compounds. Davisil Chromatographic Silica gel (200-425 mesh) was used for column chromatographic separations and Silica gel Merck, TLC grade 7749 with gypsum binder and fluorescent indicator was used for the preparation of the Chromatotron rotors. All reactions and purification procedures were monitored using Whatrnan TLC plates with a fluorescent indicator.

As noted above and shown in Scheme 5, chalcone derivatives are synthesized by the Claisen condensation of appropriately substituted and commercially available acetophenones and benzaldehydes in ethanol in the presence of 50% aqueous KOH. Corresponding substituted acetophenones were mixed with an equimolar amount of substituted benzaldehydes in ethanol. 5 milliliters of 50% aqueous KOH was added and the reaction mixture heated at 50-60° C. for 2-6 h. The product precipitated after cooling as yellow crystals, which were collected, recrystallized from CH₃0H/CH₂Cl and finally purified by column chromatography, using CH₂Cl₂:hexane 5:1 as eluent.

Synthesis of substituted chalcones: the methylated chalcone was synthesized by selective dealkylation of commercially available 2,4,6-trihydroxybenzaldehyde in the presence of aluminum tribromide using acetonitrile as the solvent. The aldehyde was reduced by means of triethylsilane in trifluoroacetic acid. Acylation of the product with acetic anhydride gave the appropriately substituted precursor for chalcone formation.

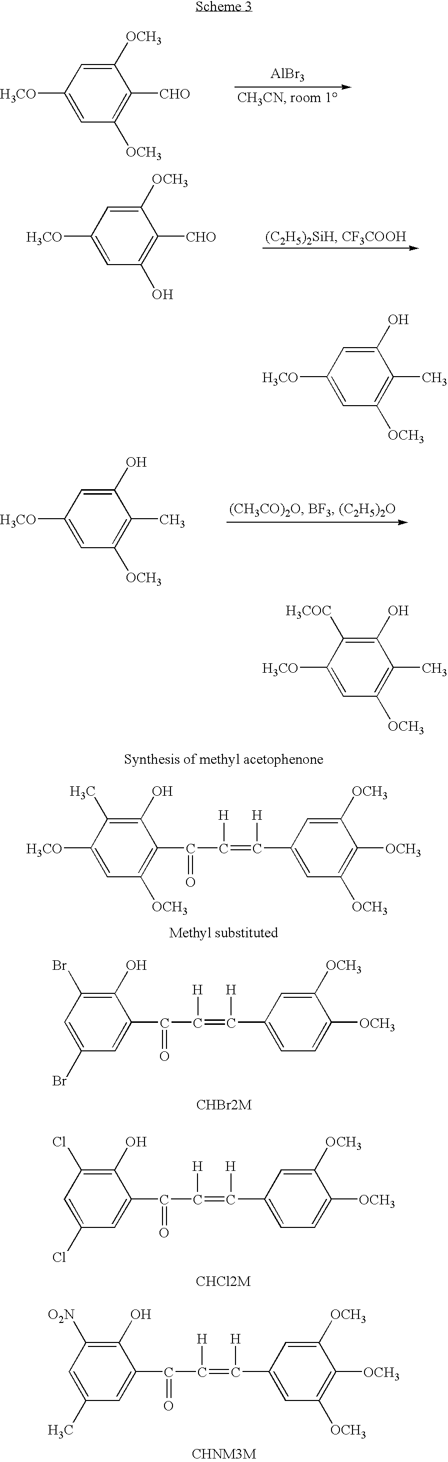

### Example 1

### Synthesis of Chalcones 5-10

Employing the general method described by N. N. Mateeva et al, J. Heterocyclic Chem., 39, 1251 (2002); D. K. Bhardwaj et al, Indian J. Chem., 27B, 261 (1988) and K. Krohn et al, Phytochemistry 61.931 (2002), The corresponding substituted acetophenones 1 were mixed with an equimolar amount of 3,4-dimethoxybenzaldehyde or 3,4,5-trimethoxybenzaldehyde, 2, in ethanol (Scheme 1). 5 Milliliters of 50% aqueous KOH was added and the reaction mixture heated at 50-60° C. for 2-6 h. The product precipitated after cooling as yellow crystals, which were collected, recrystallized from CH₃OH/CH₂Cl₂ and finally purified by column chromatography, using CH₂Cl₂:hexane 5:1 as eluent. The yields were 60-80%.

### Example 2

### Synthesis of Flavones 17-22

Employing the general method described by Mateeva, supra, T. Hone et al, Chem. Pharm. Bull., 43, 2054 (1995) and C. J. Bennett et al, Bioorganic & Medicinal Chemistry, 12, 2079 (2004). Compounds 3 (Scheme 2) were mixed with 1.7 times excess of 3,4,5-trimethoxy benzoyl chloride or 3,4-dimethoxy benzoyl chloride, 4, correspondingly, in pyridine. The reaction mixture was heated at 70° C. for 4 h, and then poured onto ice containing 5 N HCL. The precipitate that formed was collected by vacuum filtration and air dried. The crude product was dissolved in pyridine in the presence of 10 times excess of KOH.

The reaction was carried out for 4 h at 65° DC, and then the mixture poured onto an iced 5 N HCl bath. A yellow precipitate was formed, which was collected by filtration and air dried. The crude product was dissolved in acetic acid containing 1 mL concentrated H₂SO₄, heated at 60° C. for 4 h and left stirring overnight at room temperature. After 24 h, the reaction mixture was poured onto an ice-NaHC03 mixture, the product collected by filtration and recrystallized from ethanol.

The pure flavone was obtained by purification on Chromatotrone with CH₂Cl₂:MeOH 8:1 as eluent. The yields were 30-50%.

The purified and identified analogs were subjected to in vitro biological testing. The results of the testing clearly demonstrated inhibition of the H-9 cell lines (the HIV or cancer virus cell lines), as well as significant inhibition of the MCF-7 cell lines (breast cancer cells) and PC-3 cell lines (prostate cancer cell lines). See tables 3-17. The biological activities of the flavonoids were compared with the activity of AZT (a well established anti-HIV and anti-cancer drug) under the same conditions. Three compounds (CHBr2M, CHCI2M and CHNM3M) showed a much superior inhibitory activities, compared to AZT when assayed against the above mentioned cell lines under the same conditions.

TABLE I
Elemental Analysis and Melting Point Data for the Synthesized Compounds

| | | | Elemental analysis | m.p. |
|---|---|---|---|---|
| | Compound | Composition | Calculated/Found | ° C. |
| 5 | R₁═R₂═Cl, R₃═OCH₃ | C₁₈H₁₆Cl₂O₅ | C56.41 H4.21 | 145-146 |
| | | | C56.31 H4.41 | |
| 6 | R₁═R₂═Br, R₃═OCH₃ | C₁₈H₁₆Br₂O₅ | C45.79 H3.42 | 152-153 |
| | | | C45.65 H3.74 | |
| 7 | R₁═NO₂, R₂═CH₃, R═OCH₃ | C₁₉H₁₉NO₇ | C61.12 H5.13 | 147-148 |
| | | | C61.11 H5.40 | |
| 8 | R₁═R₂═Cl, R₃═H | C₁₇H₁₄Cl₂O₄ | C57.81 H4.00 | 163-164 |
| | | | C58.03 H4.39 | |
| 9 | R₁═R₂═Br, R₃═H | C₁₇H₁₄Br₂O₄ | C46.18 H3.19 | 158-159 |
| | | | C46.29 H3.43 | |
| 10 | R₁═NO₂, R₂═CH₃, R₃═H | C₁₈H₁₇NO₆ | C62.97 H4.99 | 153-154 |
| | | | C62.97 H5.33 | |
| 11 | R₁═R₂═Cl, R₃═OCH₃ | C₁₈H₁₄Cl₂O₆ | C54.43 H3.55 | 198-199 |
| | | | C54.36 H3.85 | |
| 12 | R₁═R₂═Br, R₃═OCH₃ | C₁₈H₁₄Br₂O₆ | C44.47 H2.90 | >260, |
| | | | C44.12 H3.19 | decomp |
| 13 | R₁═NO₂, R₂═CH₃, R₃═OCH₃ | C₁₉H₁₇NO₈ | C58.92 H4.42 | 210-211 |
| | | | C58.97 H4.71 | |
| 14 | R₁═R₂═Cl, R₃═H | C₁₇H₁₂Cl₂O₅ | C55.61 H3.29 | 215-216 |
| | | | C55.41 H3.45 | |
| 15 | R₁═R₂═Br, R₃═H | C₁₇H₁₂Br₂O₅ | C44.77 H2.65 | 220-221 |
| | | | C44.81 H2.99 | |
| 16 | R₁═NO₂, R₂═CH₃, R₃═H | C₁₈H₁₅NO₇ | C60.50 H4.23 | 195-196 |
| | | | C60.41 H4.21 | |
| 17 | R₁═R₂═Cl, R₃═OCH₃ | C₁₈H₁₄Cl₂O₅ | C56.71 H3.70 | 155-156 |
| | | | C57.03 H3.81 | |
| 18 | R₁═R₂═Br, R₃═OCH₃ | C₁₈H₁₄Br₂O₅ | C45.99 H3.00 | 179-180 |
| | | | C46.28 H3.39 | |
| 19 | R₁═NO₂, R₂═CH₃, R₃═OCH₃ | C₁₉H₁₇NO₇ | C61.45 H4.61 | 135-136 |
| | | | C61.47 H5.01 | |
| 20 | R₁═R₂═Cl, R₃═H | C₁₇H₁₂Cl₂O₄ | C58.14 H3.44 | 145-146 |
| | | | C58.37 H3.76 | |
| 21 | R₁═R₂═Br, R₃═H | C₁₇H₁₂Cl₂O₄ | C46.40 H2.75 | 134-135 |
| | | | C46.72 H3.12 | |
| 22 | R₁═NO₂, R₂═CH₃, R₃═H | C₁₈H₁₅NO₆ | C63.34 H4.43 | 150-151 |
| | | | C62.95 H4.80 | |

TABLE 2
NMR and IR Data for the Synthesized Compounds

| Compound | ¹H-NMR (δ ppm) | IR (v cm⁻¹ KBr) |
|---|---|---|
| 5 | (DMSO-d₆): δ 3.70, 3.79 (s, s, 9H, —CH₃O), | 3400 (broad, OH), |
| | 6.82 (s, 2H, Ar), 7.07-7.08 (d, 1H, Ar, J = 3.0 | 1590 (C═O), |
| | Hz), 7.42-7.43 (d, 1H, Ar, J = 3.0 Hz), | 1610 (C═C Ar), |
| | 7.37-7.42 (d, 1H, CH═CH, J = 15 Hz) | 1590 (C═C), |
| | 8.55-8.60 (d, 1H, CH═CH, J = 15 Hz) | 1130 (C—O) cm⁻¹. |
| 6 | (DMSO-d₆): δ 3.78, 3.80 (s, s, 9H, —CH₃O), | 3390 (broad, OH), |
| | 7.14 (s, 2H, Ar), 7.34-7.35 (d, 1H, J = 3.0 Hz), | 1585 (C═O), |
| | 7.39-7.45 (d, 1H, J = 18 Hz, CH═CH), 7.58-7.59 | 1600 (C═C Ar), |
| | (d, 1H, J = 3.0 Hz), 8.48-8.53 (d, 1H, J = 18 Hz) | 1595 (C═C), |
| | | 1110 (C—O) cm⁻¹. |
| 7 | (DMSO-d₆): δ 3.81, 3.84 (s, s, 9H, —CH₃O), | 3390 (broad, OH), |
| | 6.87 (s, 2H, Ar), 7.37-7.43 (d, 1H, J = 18 Hz | 1640 (C═O), |
| | CH═CH), 7.48-7.49 (d, 1H, J = 3.0 Hz, Ar), | 1612 (C═C Ar), |
| | 8.08-8.14 (d, 1H, J = 18 Hz, CH═CH) | 1580 (C═C), |
| | | 1120 (C—O) cm⁻¹. |
| 8 | (DMSO-d₆): δ 3.93, 3.97 (s, s, 6H, —CH₃O), | 3400 (broad, OH), |
| | 6.88-6.92 (m, 1H, Ar), 7.15-7.16 (d, 1H, J = | 1650 (C═O), |
| | 3.0 Hz, Ar), 7.24-7.29 (m, 1H, Ar), 7.34-7.39 (d, | 1610 (C═C Ar), |
| | 1H, J = 15 Hz, CH═CH), 7.54-7.57 (m, 1H, Ar), | 1590 (C═C), |
| | 7.80-7.81 (d, 1H, J = 3.0 Hz, Ar), 7.91-7.96 (d, 1H, | 1125 (C—O) cm⁻¹. |
| | J = 15 Hz, CH═CH) | |
| 9 | (DMSO-d₆): δ 3.07, 3.10 (s, s, 6H, —CH₃O), | 3390 (broad, OH). |
| | 6.88-6.92 (m, 1H, Ar), 7.15-7.16 (d, 1H, Ar), | 1650 (C═O), |
| | 7.26-7.30 (dd, 1H, Ar), 7.34-7.39 (d, 1H, J = 15 | 1611 (C═C Ar), |
| | Hz, CH═CH), 7.86-7.91 (d, 1H, J = 15 Hz, | 1590 (C═C), |
| | CH═CH), 7.94-7.98 (m, 1H, Ar) | 1110 (C—O) cm⁻¹. |
| 10 | (DMSO-d₆): δ 3.77, 3.80 (s, s, 6H, —CH₃O), | 3390 (broad, OH), |
| | 6.95-6.98 (d, 1H, J = 9 Hz), 7.17 (s, 1H, Ar), | 1640 (C═O), |
| | 7.22-7.23 (d, 1H, J = 3.0 Hz, Ar), 7.40-7.45 (d, 1H, | 1610 (C═C Ar), |
| | J = 15 Hz, CH═CH), 7.65-7.66 (d, 1H, J = 3.0 Hz, | 1592 (C═C), |
| | Ar), 798-8.03 (d, 1H, J = 15 Hz, CH═CH) | 1120 (C—O) cm⁻¹. |
| 11 | (DMSO-d₆): δ 3.68, 3.81 (s, s, 9H, —CH₃O), | 3400 (broad, OH), |
| | 7.83-7.84 (d, 1H, Ar, J = 2.4 Hz), 7.85-7.86 (d, 1H, | 1650 (C═O), |
| | Ar, J = 2.4 Hz), 8.08 (s, 2H, Ar) | 1613 (C═C Ar), |
| | | 1590 (C═C), |
| | | 1131 (C—O) cm⁻¹. |
| 12 | (DMSO-d₆): δ 3.68, 3.81 (s, s, 9H, —CH₃O), | 3390 (broad, OH), |
| | 7.98-7.99 (d, 1H, Ar, J = 2.4 Hz), 8.01-8.02 (d, 1H, | 1655 (C═O), |
| | Ar, J = 2.4 Hz), 8.19 (s, 2H, Ar) | 1605 (C═C Ar), |
| | | 1580 (C═C), |
| | | 1115 (C—O) cm⁻¹. |
| 13 | (DMSO-d₆): δ 3.76, 3.86 (s, s, 6H, —OCH₃), | 3410 (broad, OH). |
| | 7.69 (s, 2H, Ar), 8.25-8.26 (d, 1H, J = 3.0 Hz, Ar), | 1660 (C═O), |
| | 8.42-8.43 (d, 1H, J = 3.0 Hz, Ar). | 1613 (C═C Ar), |
| | | 1595 (C═C), |
| | | 1115 (C—O) cm⁻¹. |
| 14 | (DMSO-d₆): δ 3.79 (s, 6H, —OCH₃), 7.01-7.04 | 3380 (broad, OH), |
| | (d, 1H, Ar, J = 9.0 Hz), 7.81 (s, 1H, Ar), | 1645 (C═O), |
| | 7.87 (m, 1H, Ar), 8.12-8.15 (d, 1H, Ar, J = 9.0 Hz), | 1610 (C═C Ar), |
| | 8.44 (s, 1H, Ar) | 1595 (C═C), |
| | | 1120 (C—O) cm⁻¹. |
| 15 | (DMSO-d₆): δ 3.29, 3.31 (s, s, 6H, —OCH₃), | 3400 (broad, OH), |
| | 7.17-7.19 (d, 1H, Ar, J = 6 Hz), 7.88-7.98 | 1656 (C═O), |
| | (m, 2H, Ar), 8.14-8.15 (d, 1H, J = 3.0 Hz), | 1600 (C═C Ar), |
| | 8.34-8.35 (d, 1H, Ar, J = 3.0 Hz) | 1595 (C═C), |
| | | 1130 (C—O) cm⁻¹. |
| 16 | (DMSO-d₆): δ 3.77-3.80 (s, s, 6H, —OCH₃), | 3380 (broad, OH), |
| | 6.97-6.99 (m, 2H, Ar), 8.16 (s, 1H, Ar), | 1660 (C═O), |
| | 8.33-8.35 (dd, J = 6 Hz, Ar), 8.48 (s, 1H, Ar) | 1610 (C═C Ar), |
| | | 1588 (C═C), |
| | | 1130 (C—O) cm⁻¹. |
| 17 | (deuteriochloroform): δ 3.87, 3.89 (s, 9H, —CH₃O), | 1660 (C═O), |
| | 8.01-8.02 (d, J = 2.4 Hz, 1H, Ar), 7.67-7.68 | 1613 (C═C Ar), |
| | (d, J = 2.4 Hz, 1H, Ar), 6.74 (s, 1H, O═C—CH═), | 1592 (C═C), |
| | 7.14 (s, 2H, Ar) | 1133 (C—O) cm⁻¹. |
| 18 | (deuteriochloroform): δ 3.93, 3.94 (s, 9H, —CH₃O), | 1656 (C═O), |
| | 8.27-8.28 (d, J = 2.2 Hz, 1H, Ar), 8.02-8.03 | 1610 (C═C Ar), |
| | (d, J = 2.2 Hz, 1H, Ar), 6.80 (s, 1H, O═C—CH═), | 1591 (C═C), |
| | 7.23 (s, 2H, Ar) | 1134 (C—O) cm⁻¹. |
| 19 | (deuteriochloroform): δ 3.87, 3.91 (s, 9H, —CH₃O), | 1654 (C═O), |
| | 8.24 (s, 1H, Ar), 8.19-8.20 (d, J = 2.1 Hz, | 1618 (C═C Ar), |
| | 1H, Ar), 6.79 (s, 1H, O═C—CH═), 7.23 (s, 2H, Ar), | 1583 (C═C), |
| | 2.49 (s, 3H, —CH₃) | 1126 (C—O) cm⁻¹. |
| 20 | (deuteriochloroform): δ 3.95, 3.96 (s, 6H, —CH₃O), | 1654 (C═O), |
| | 8.07-8.06 (d, J = 2.3 Hz, 1H, Ar), 7.70-7.71 | 1561 (C═C Ar), |
| | (d, J = 2.6 Hz, 1H, Ar), 6.77 (s, 1H, O═C—CH═), | 1519 (C═C), |
| | 6.97-6.99 (d, J = 8.4 Hz, 1H, Ar) 7.44-7.45 | 1278 (C—O) cm⁻¹. |
| | (d, J = 2.0 Hz, 1H, Ar), (td, J = 2.3, 8.7 Hz, 1H, Ar) | |
| 21 | (deuteriochloroform): δ 3.92, 3.93 (s, 6H, —CH₃O), | 1677 (C═O), |
| | 8.26-8.27 (d, J = 2.4 Hz, 1H, Ar), 7.99-8.00 | 1650 (C═C Ar), |
| | (d, J = 2.2 Hz, 1H, Ar), 6.82 (s, 1H, O═C—CH═), | 1599 (C═C), |
| | 7.74-7.77 (dd, J = 1.9 Hz, 6.4 Hz, 1H, Ar), | 1235 (C—O) cm⁻¹. |
| | 7.57-7.58 (d, J = 1.6 Hz, 1H, Ar), 6.88-6.91 (d, | |
| | J = 8.5 Hz, 1H, Ar) | |
| 22 | (deuteriochloroform): δ 3.96, 4.00 (s, 6H, —CH₃O), | 1647 (C═O), |
| | 8.30 (s, 1H, Ar), 8.22-8.23 (d, J = 2.0 Hz, | 1615 (C═C Ar), |
| | 1H, Ar), 6.84 (s, 1H, O═C—CH═), 6.97-7.00 (d, | 1531 (C═C), |
| | J = 8.9 Hz, 1H, Ar), 7.82-8.00 (dd, J = 1.8 Hz, 50.3, | 1256 (C—O) cm⁻¹. |
| | 1H, Ar), 7.58-7.59 (d, J = 2.0 Hz, 1H, Ar), 2.54 (s, | |
| | 3H, —CH₃) | |

TABLE 3

| K. K. Redda/Chavor | Cytotoxicity on H-9 cells | | | | 12-05 to | |
|---|---|---|---|---|---|---|
| Exp.: H-9 cells | Cells | | | % | 12-09/2005 | |
| Drugs | (×10⁴) | Mean | SD | Inhibition | Mean | SD |
| | 49.5 | | | | | |
| 2% DMSO | 50.5 | 50.25 | 0.54 | 0 | | |
| | 50.8 | | | | | |
| AZT (μg/ml) | 41.3 | | | 17.91 | | |
| 1 | 37.5 | 40.83 | 2.57 | 25.37 | 18.74 | 5.11 |
| | 43.8 | | | 12.94 | | |
| | 30.5 | | | 39.30 | | |
| 5 | 30.0 | 30.92 | 0.96 | 40.30 | 38.47 | 1.92 |
| | 32.3 | | | 35.82 | | |
| | 26.0 | | | 48.26 | | |
| 10 | 25.0 | 25.25 | 0.54 | 50.25 | 49.75 | 1.07 |
| | 24.8 | | | 50.75 | | |
| CHBr 2M (μg/ml) | 45.5 | | | 9.45 | | |
| 1 | 49.5 | 47.42 | 1.64 | 1.49 | 5.64 | 3.26 |
| | 47.3 | | | 5.97 | | |
| | 25.5 | | | 49.25 | | |
| 5 | 24.8 | 25.33 | 0.42 | 50.75 | 49.59 | 0.85 |
| | 25.8 | | | 48.76 | | |
| | 18.3 | | | 63.68 | | |
| 10 | 16.3 | 17.58 | 0.94 | 67.68 | 65.01 | 1.88 |
| | 18.3 | | | 63.68 | | |
| | 11.8 | | | 76.62 | | |
| 50 | 11.5 | 11.58 | 0.12 | 77.11 | 76.95 | 0.23 |
| | 11.5 | | | 77.11 | | |
| CHCl 2M (μg/ml) | 42.0 | | | 16.42 | | |
| 1 | 42.3 | 42.25 | 0.20 | 15.92 | 15.92 | 0.41 |
| | 42.5 | | | 15.42 | | |
| | 24.0 | | | 52.24 | | |
| 5 | 23.8 | 24.00 | 0.20 | 52.74 | 52.24 | 0.41 |
| | 24.3 | | | 51.74 | | |
| | 13.0 | | | 74.13 | | |
| 10 | 11.5 | 12.92 | 1.12 | 77.11 | 74.30 | 2.24 |
| | 14.3 | | | 71.64 | | |
| | 4.0 | | | 92.04 | | |
| 50 | 4.5 | 4.50 | 0.41 | 91.04 | 91.04 | 0.81 |
| | 5.0 | | | 90.05 | | |
| CHNM 2M (μg/ml) | 57.3 | | | −13.93 | | |
| 1 | 60.3 | 58.17 | 1.48 | −19.90 | −15.75 | 2.94 |
| | 57.0 | | | −13.43 | | |
| | 61.5 | | | −22.39 | | |
| 5 | 55.5 | 58.58 | 2.45 | −10.45 | −16.58 | 4.88 |
| | 58.8 | | | −16.92 | | |
| | 50.5 | | | −0.50 | | |
| 10 | 52.3 | 50.75 | 1.14 | −3.98 | −1.00 | 2.26 |
| | 49.5 | | | 1.49 | | |
| | 56.8 | | | −12.94 | | |
| 50 | 54.8 | 56.00 | 0.89 | −8.96 | −11.44 | 1.77 |
| | 56.5 | | | −12.44 | | |
| H-9 | mean | | | | | |

TABLE 6

| K. K. Redda/Chavor | Cytotoxicity on MCF-7 cells | | | | 11-14 to | |
|---|---|---|---|---|---|---|
| Exp.: MCF-7 cells | Cells | | | % | 11-18/2005 | |
| Drugs | (×10⁴) | Mean | SD | Inhibition | Mean | SD |
| | 56.8 | | | | | |
| 2% DMSO | 61.8 | 57.83 | 2.86 | 0 | | |
| | 55.0 | | | | | |
| AZT (μg/ml) | 53.0 | | | 8.36 | | |
| 1 | 55.3 | 53.83 | 1.01 | 4.47 | 6.92 | 1.74 |
| | 53.3 | | | 7.93 | | |
| | 34.8 | | | 39.91 | | |
| 5 | 34.5 | 35.50 | 1.24 | 40.35 | 38.62 | 2.15 |
| | 37.3 | | | 35.59 | | |
| | 22.3 | | | 61.53 | | |
| 10 | 23.0 | 22.50 | 0.35 | 60.23 | 61.10 | 0.61 |
| | 22.3 | | | 61.53 | | |
| CHBr 2M (μg/ml) | 48.8 | | | 15.71 | | |
| 1 | 51.5 | 49.08 | 1.85 | 10.95 | 15.13 | 3.20 |
| | 47.0 | | | 18.73 | | |
| | 27.0 | | | 53.31 | | |
| 5 | 25.3 | 26.25 | 0.74 | 56.34 | 54.61 | 1.27 |
| | 26.5 | | | 54.18 | | |
| | 7.3 | | | 87.46 | | |
| 10 | 9.0 | 7.92 | 0.77 | 84.44 | 86.31 | 1.34 |
| | 7.5 | | | 87.03 | | |
| | 0.8 | | | 98.70 | | |
| 50 | 1.0 | 0.75 | 0.20 | 98.27 | 98.70 | 0.35 |
| | 0.5 | | | 99.14 | | |
| CHCl 2M (μg/ml) | 49.8 | | | 13.98 | | |
| 1 | 48.5 | 49.42 | 0.66 | 16.14 | 14.55 | 1.13 |
| | 50.0 | | | 13.54 | | |
| | 32.0 | | | 44.67 | | |
| 5 | 31.5 | 32.33 | 0.85 | 45.53 | 44.09 | 1.47 |
| | 33.5 | | | 42.07 | | |
| | 10.0 | | | 82.71 | | |
| 10 | 10.8 | 10.42 | 0.31 | 81.41 | 81.99 | 0.54 |
| | 10.5 | | | 81.84 | | |
| | 1.0 | | | 98.27 | | |
| 50 | 0.5 | 0.75 | 0.20 | 99.14 | 98.70 | 0.35 |
| | 0.8 | | | 98.70 | | |
| CHNM 2M (μg/ml) | 53.0 | | | 8.36 | | |
| 1 | 55.3 | 53.92 | 0.96 | 4.47 | 6.77 | 1.67 |
| | 53.5 | | | 7.49 | | |
| | 45.5 | | | 21.33 | | |
| 5 | 45.0 | 46.17 | 1.31 | 22.19 | 20.17 | 2.27 |
| | 48.0 | | | 17.00 | | |
| | 40.5 | | | 29.97 | | |
| 10 | 39.0 | 39.75 | 0.61 | 32.56 | 31.27 | 1.06 |
| | 39.8 | | | 31.27 | | |
| | 32.5 | | | 43.80 | | |
| 50 | 34.5 | 33.50 | 0.82 | 40.35 | 42.07 | 1.41 |
| | 33.5 | | | 42.07 | | |
| MCF-7 | mean | | | | | |

TABLE 7

| K. K. Redda/Chavor | Cytotoxicity on PC-3 cells | | | | 11-11 to | |
|---|---|---|---|---|---|---|
| Exp.: PC-3 cells | Cells | | | % | 11-17/2005 | |
| Drugs | (×10⁴) | Mean | SD | Inhibition | Mean | SD |
| | 99.3 | | | | | |
| 2% DMSO | 98.3 | 98.00 | 1.14 | 0 | | |
| | 96.5 | | | | | |
| AZT (μg/ml) | 84.0 | | | 14.29 | | |
| 1 | 85.5 | 84.58 | 0.56 | 12.76 | 13.69 | 0.67 |
| | 84.3 | | | 14.03 | | |
| | 70.5 | | | 28.06 | | |
| 5 | 72.3 | 71.33 | 0.72 | 26.28 | 27.21 | 0.73 |
| | 71.3 | | | 27.30 | | |
| | 61.5 | | | 37.24 | | |
| 10 | 61.3 | 61.58 | 0.31 | 37.50 | 37.16 | 0.32 |
| | 62.0 | | | 38.73 | | |
| CM-091303 (μg/ml | 73.3 | | | 25.26 | | |
| 1 | 72.0 | 70.92 | 2.47 | 26.53 | 27.64 | 2.52 |
| | 67.5 | | | 31.12 | | |
| | 75.5 | | | 22.96 | | |
| 5 | 68.0 | 70.83 | 3.32 | 30.61 | 27.72 | 3.39 |
| | 69.0 | | | 29.59 | | |
| | 77.5 | | | 20.92 | | |
| 10 | 70.0 | 72.25 | 3.72 | 28.57 | 26.28 | 3.80 |
| | 69.3 | | | 29.34 | | |
| | 57.5 | | | 41.33 | | |
| 50 | 53.0 | 56.00 | 2.12 | 45.92 | 42.86 | 2.16 |
| | 57.5 | | | 41.33 | | |
| CM-090803 (μg/ml | 74.3 | | | 24.23 | | |
| 1 | 74.0 | 73.42 | 1.01 | 24.49 | 25.09 | 1.03 |
| | 72.0 | | | 26.53 | | |
| | 69.0 | | | 29.59 | | |
| 5 | 69.5 | 70.00 | 1.08 | 29.08 | 28.57 | 1.10 |
| | 71.5 | | | 27.04 | | |
| | 68.0 | | | 30.61 | | |
| 10 | 70.0 | 69.58 | 1.16 | 28.57 | 29.00 | 1.18 |
| | 70.8 | | | 27.81 | | |
| | 67.3 | | | 31.38 | | |
| 50 | 65.5 | 62.08 | 6.11 | 33.16 | 35.65 | 6.24 |
| | 53.5 | | | 45.41 | | |
| CM-110104 (μg/ml | 88.5 | | | 9.69 | | |
| 1 | 81.3 | 89.08 | 6.65 | 17.09 | 9.10 | 6.78 |
| | 97.5 | | | 0.51 | | |
| | 59.5 | | | 39.29 | | |
| 5 | 61.8 | 62.67 | 3.03 | 36.99 | 36.05 | 3.09 |
| | 66.8 | | | 31.89 | | |
| | 62.5 | | | 36.22 | | |
| 10 | 60.3 | 62.92 | 2.37 | 38.52 | 35.80 | 2.41 |
| | 68.0 | | | 32.65 | | |
| | 49.8 | | | 49.23 | | |
| 50 | 52.5 | 50.17 | 1.76 | 46.43 | 48.81 | 1.80 |
| | 48.3 | | | 50.77 | | |
| PC-3 | mean | | | | | |

TABLE 10

| K. K. Redda/Chavor | Cytotoxicity on MCF-7 cells | | | | 9-26 to | |
|---|---|---|---|---|---|---|
| Exp.: MCF-7 cells | Cells | | | % | 9-30/2005 | |
| Drugs | (×10⁴) | Mean | SD | Inhibition | Mean | SD |
| | 49.0 | | | | | |
| 2% DMSO | 50.5 | 49.58 | 0.66 | 0 | | |
| | 49.3 | | | | | |
| AZT (μg/ml) | 35.5 | | | 28.40 | | |
| 1 | 36.8 | 36.42 | 0.66 | 25.88 | 26.55 | 1.32 |
| | 37.0 | | | 25.38 | | |
| | 24.8 | | | 49.98 | | |
| 5 | 27.3 | 26.35 | 1.10 | 45.04 | 46.86 | 2.22 |
| | 27.0 | | | 45.55 | | |
| | 17.3 | | | 65.21 | | |
| 10 | 18.3 | 17.25 | 0.82 | 63.19 | 65.21 | 1.65 |
| | 16.3 | | | 67.23 | | |
| CM-091303 (μg/ml | 35.8 | | | 27.90 | | |
| 1 | 35.8 | 36.17 | 0.59 | 27.90 | 27.05 | 1.19 |
| | 37.0 | | | 25.38 | | |
| | 35.3 | | | 28.91 | | |
| 5 | 36.8 | 35.75 | 0.71 | 25.88 | 27.90 | 1.43 |
| | 35.3 | | | 28.91 | | |
| | 29.8 | | | 40.00 | | |
| 10 | 29.3 | 30.17 | 0.96 | 41.01 | 39.16 | 1.95 |
| | 31.5 | | | 36.47 | | |
| | 26.3 | | | 47.06 | | |
| 50 | 26.8 | 26.17 | 0.51 | 46.05 | 47.23 | 1.04 |
| | 25.5 | | | 48.57 | | |
| CM-090803 (μg/ml | 33.8 | | | 31.93 | | |
| 1 | 33.5 | 33.33 | 0.42 | 32.44 | 32.77 | 0.86 |
| | 32.8 | | | 33.95 | | |
| | 26.0 | | | 47.56 | | |
| 5 | 29.3 | 28.17 | 1.53 | 41.01 | 43.19 | 3.09 |
| | 29.3 | | | 41.01 | | |
| | 28.8 | | | 42.02 | | |
| 10 | 28.3 | 28.58 | 0.24 | 43.03 | 42.35 | 0.48 |
| | 28.8 | | | 42.02 | | |
| | 23.3 | | | 53.11 | | |
| 50 | 24.0 | 23.75 | 0.35 | 51.60 | 52.10 | 0.71 |
| | 24.0 | | | 51.60 | | |
| CM-110104 (μg/ml | 34.0 | | | 31.43 | | |
| 1 | 35.0 | 34.42 | 0.42 | 29.41 | 30.59 | 0.86 |
| | 34.3 | | | 30.92 | | |
| | 27.5 | | | 44.54 | | |
| 5 | 28.5 | 28.08 | 0.42 | 42.52 | 43.36 | 0.86 |
| | 28.3 | | | 43.03 | | |
| | 28.5 | | | 42.52 | | |
| 10 | 27.0 | 27.92 | 0.66 | 45.55 | 43.70 | 1.32 |
| | 28.3 | | | 43.03 | | |
| | 19.8 | | | 60.17 | | |
| 50 | 20.0 | 19.67 | 0.31 | 59.66 | 60.34 | 0.53 |
| | 19.3 | | | 61.18 | | |
| MCF-7 | mean | | | | | |

TABLE 12

| K. K. Redda/Chavor | Cytotoxicity on MCF-7 cells | | | | 9-12 to | |
|---|---|---|---|---|---|---|
| Exp.: MCF-7 cells | Cells | | | % | 9-16/2005 | |
| Drugs | (×10⁴) | Mean | SD | Inhibition | Mean | SD |
| | 60.5 | | | | | |
| 2% DMSO | 55.5 | 57.67 | 2.09 | 0 | | |
| | 57.0 | | | | | |
| AZT (μg/ml) | 44.5 | | | 22.83 | | |
| 1 | 46.0 | 45.25 | 0.61 | 20.23 | 21.53 | 1.06 |
| | 45.3 | | | 21.53 | | |
| | 30.0 | | | 47.98 | | |
| 5 | 27.8 | 28.58 | 1.01 | 51.88 | 50.43 | 1.75 |
| | 28.0 | | | 51.45 | | |
| | 17.5 | | | 69.65 | | |
| 10 | 21.5 | 20.17 | 1.89 | 62.72 | 65.03 | 3.27 |
| | 21.5 | | | 62.72 | | |
| M/R-63-1 (μg/ml) | 33.3 | | | 42.34 | | |
| 1 | 32.6 | 32.17 | 1.20 | 43.21 | 44.22 | 2.07 |
| | 30.5 | | | 47.11 | | |
| | 27.3 | | | 52.75 | | |
| 5 | 27.5 | 28.42 | 1.48 | 52.31 | 50.72 | 2.56 |
| | 30.5 | | | 47.11 | | |
| | 30.8 | | | 46.68 | | |
| 10 | 29.3 | 29.25 | 1.22 | 49.28 | 49.28 | 2.12 |
| | 27.8 | | | 51.88 | | |
| | 24.3 | | | 57.95 | | |
| 50 | 23.5 | 23.58 | 0.51 | 59.25 | 59.10 | 0.89 |
| | 23.0 | | | 60.12 | | |
| M/R-67 (μg/ml) | 39.3 | | | 31.94 | | |
| 1 | 38.8 | 39.67 | 0.96 | 32.80 | 31.21 | 1.67 |
| | 41.0 | | | 28.90 | | |
| | 38.3 | | | 33.67 | | |
| 5 | 37.5 | 37.67 | 0.42 | 34.97 | 34.68 | 0.74 |
| | 37.3 | | | 35.40 | | |
| | 36.0 | | | 37.57 | | |
| 10 | 35.8 | 35.17 | 1.01 | 38.01 | 39.02 | 1.75 |
| | 33.8 | | | 41.47 | | |
| | 30.5 | | | 47.11 | | |
| 50 | 31.5 | 30.50 | 0.82 | 45.38 | 47.11 | 1.42 |
| | 29.5 | | | 48.84 | | |
| M/R-64-1 (μg/ml) | 30.8 | | | 46.68 | | |
| 1 | 27.5 | 29.08 | 1.33 | 52.31 | 49.57 | 2.30 |
| | 29.0 | | | 49.71 | | |
| | 29.0 | | | 49.71 | | |
| 5 | 28.0 | 29.00 | 0.82 | 51.45 | 49.71 | 1.42 |
| | 30.0 | | | 47.98 | | |
| | 27.0 | | | 53.18 | | |
| 10 | 30.0 | 29.08 | 1.48 | 47.98 | 49.57 | 2.56 |
| | 30.3 | | | 47.54 | | |
| | 29.8 | | | 48.41 | | |
| 50 | 28.0 | 29.00 | 0.74 | 51.45 | 49.71 | 1.28 |
| | 29.3 | | | 49.28 | | |
| M/R-68-1 (μg/ml) | 50.3 | | | 12.86 | | |

TABLE 14

| K. K. Redda/Chavor | Cytotoxicity on MCF-7 cells | | | | 9-08 to | |
|---|---|---|---|---|---|---|
| Exp.: MCF-7 cells | Cells | | | % | 9-12/2005 | |
| Drugs | (×10⁴) | Mean | SD | Inhibition | Mean | SD |
| | 74.3 | | | | | |
| 2% DMSO | 68.5 | 70.50 | 2.65 | 0 | | |
| | 68.8 | | | | | |
| AZT (μg/ml) | 51.0 | | | 27.66 | | |
| 1 | 53.8 | 52.33 | 1.12 | 23.76 | 25.77 | 1.59 |
| | 52.3 | | | 25.89 | | |
| | 42.3 | | | 40.07 | | |
| 5 | 42.3 | 42.75 | 0.71 | 40.07 | 39.36 | 1.00 |
| | 43.8 | | | 37.94 | | |
| | 30.0 | | | 57.45 | | |
| 10 | 30.0 | 30.00 | 0.00 | 57.45 | 57.45 | 0.00 |
| | 30.0 | | | 57.45 | | |
| M/R-67 (μg/ml) | 46.8 | | | 33.69 | | |
| 1 | 43.8 | 44.92 | 1.31 | 37.94 | 36.29 | 1.86 |
| | 44.3 | | | 37.23 | | |
| | 41.8 | | | 40.78 | | |
| 5 | 37.0 | 39.75 | 2.01 | 47.52 | 43.62 | 2.85 |
| | 40.5 | | | 42.55 | | |
| | 33.3 | | | 52.84 | | |
| 10 | 34.8 | 34.33 | 0.77 | 50.71 | 51.30 | 1.10 |
| | 35.0 | | | 50.35 | | |
| | 38.5 | | | 45.39 | | |
| 50 | 41.8 | 40.67 | 1.53 | 40.78 | 42.32 | 2.17 |
| | 41.8 | | | 40.78 | | |
| M/R-68-1 (μg/ml) | 62.3 | | | 11.70 | | |
| 1 | 65.8 | 59.75 | 2.86 | 20.92 | 15.25 | 4.05 |
| | 61.3 | | | 13.12 | | |
| | 51.0 | | | 27.66 | | |
| 5 | 44.3 | 47.92 | 2.79 | 37.23 | 32.03 | 3.95 |
| | 48.5 | | | 31.21 | | |
| | 49.5 | | | 29.79 | | |
| 10 | 51.0 | 51.42 | 1.76 | 27.66 | 27.07 | 2.50 |
| | 53.8 | | | 23.76 | | |
| | 38.8 | | | 45.04 | | |
| 50 | 36.3 | 38.92 | 2.25 | 48.58 | 44.80 | 3.19 |
| | 41.8 | | | 40.78 | | |

| MCF-7 | mean | | | | |
|---|---|---|---|---|---|
| Drugs (μg/ml) | 0 | 1 | 5 | 10 | 50 |
| AZT | | 25.77 | 39.36 | 57.45 | |
| M/R-67 | | 36.29 | 43.62 | 51.3 | 42.32 |
| M/R-68-1 | | 15.25 | 32.03 | 27.07 | 44.80 |
| SD | | | | | |

| Drugs (μg/ml) | 1 | 5 | 10 | 50 |
|---|---|---|---|---|
| AZT | 1.59 | 1 | 0 | |
| M/R-87 | 1.86 | 2.85 | 1.1 | 2.17 |

TABLE 17

| K. K. Redda/Chavor | Cytotoxicity on MCF-7 cells | | | | 12-12 to | |
|---|---|---|---|---|---|---|
| Exp.: MCF-7 cells | Cells | | | % | 12-16/2005 | |
| Drugs | (×10⁴) | Mean | SD | Inhibition | Mean | SD |
| | 50.3 | | | | | |
| 2% DMSO | 52.0 | 50.92 | 0.77 | 0 | | |
| | 50.5 | | | | | |
| AZT (μg/ml) | 43.3 | | | 15.06 | | |
| 1 | 43.3 | 43.08 | 0.24 | 15.06 | 15.38 | 0.46 |
| | 42.8 | | | 16.04 | | |
| | 31.5 | | | 38.13 | | |
| 5 | 32.3 | 32.08 | 0.42 | 36.66 | 36.99 | 0.83 |
| | 32.5 | | | 36.17 | | |
| | 22.0 | | | 56.79 | | |
| 10 | 25.5 | 24.08 | 1.50 | 49.92 | 52.70 | 2.96 |
| | 24.8 | | | 51.39 | | |
| CHCl 3M (μg/ml) | 54.0 | | | −6.06 | | |
| 1 | 54.3 | 53.17 | 1.36 | −6.55 | −4.42 | 2.67 |
| | 51.3 | | | −0.65 | | |
| | 53.8 | | | −5.56 | | |
| 5 | 51.5 | 52.75 | 0.94 | −1.15 | −3.60 | 1.84 |
| | 53.0 | | | −4.09 | | |
| | 42.5 | | | 16.53 | | |
| 10 | 43.3 | 43.75 | 1.27 | 15.06 | 14.08 | 2.50 |
| | 45.5 | | | 10.64 | | |
| | 32.0 | | | 37.15 | | |
| 50 | 32.8 | 31.50 | 1.27 | 35.68 | 38.13 | 2.50 |
| | 29.8 | | | 41.57 | | |
| CHNM 3M (μg/ml) | 44.3 | | | 13.09 | | |
| 1 | 45.0 | 44.50 | 0.35 | 11.62 | 12.60 | 0.69 |
| | 44.3 | | | 13.09 | | |
| | 25.8 | | | 49.43 | | |
| 5 | 24.0 | 24.75 | 0.74 | 52.86 | 51.39 | 1.45 |
| | 24.5 | | | 51.88 | | |
| | 5.0 | | | 90.18 | | |
| 10 | 4.8 | 4.92 | 0.12 | 90.67 | 90.34 | 0.23 |
| | 5.0 | | | 90.18 | | |
| | 1.8 | | | 96.56 | | |
| 50 | 2.0 | 1.75 | 0.20 | 96.07 | 96.56 | 0.40 |
| | 1.5 | | | 97.05 | | |

| MCF-7 | | | | | |
|---|---|---|---|---|---|
| Drugs (μg/ml) | mean | 1 | 5 | 10 | 50 |
| CHCl 3M (μg/ml) | | −4.42 | −3.6 | 14.08 | 38.13 |
| CHNM 3M (μg/ml) | | 12.6 | 51.39 | 90.34 | 96.56 |
| AZT (μg/ml) | | 15.38 | 36.99 | 52.7 | |
| SD | | | | | |
| Drugs (μg/ml) | | 1 | 5 | 10 | 50 |
| CHCl 3M (μg/ml) | | 2.67 | 1.84 | 2.5 | 2.50 |
| CHNM 3M (μg/ml) | | 0.69 | 1.45 | 0.23 | 0.40 |
| AZT (μg/ml) | | 0.46 | 0.83 | 2.96 | |

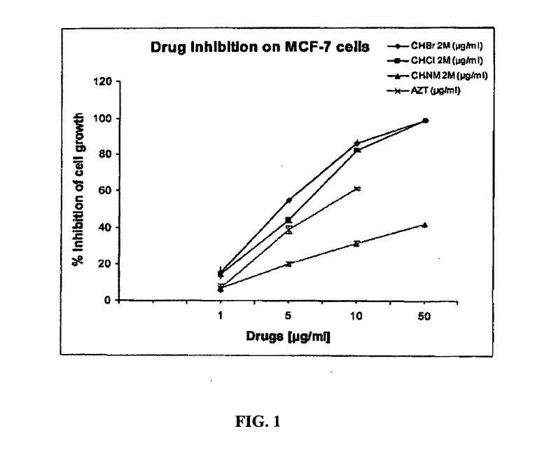

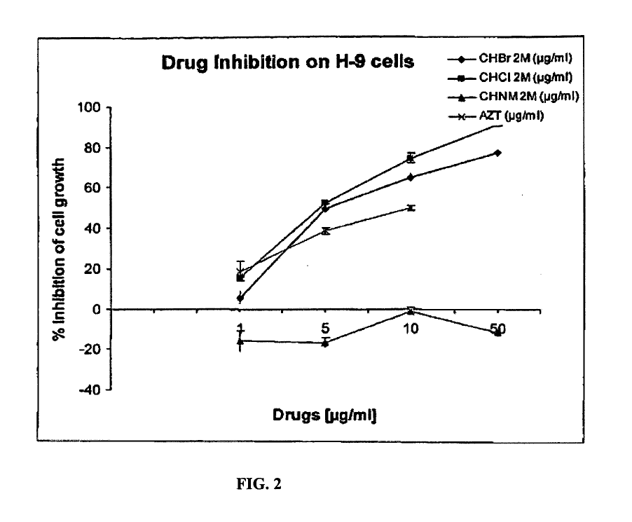

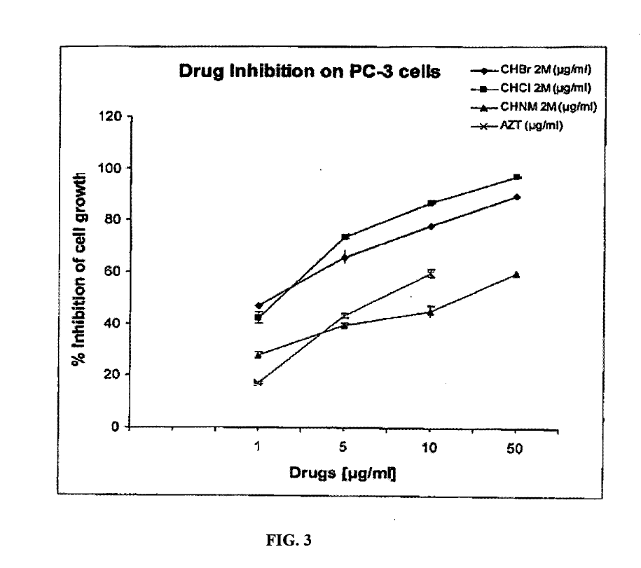

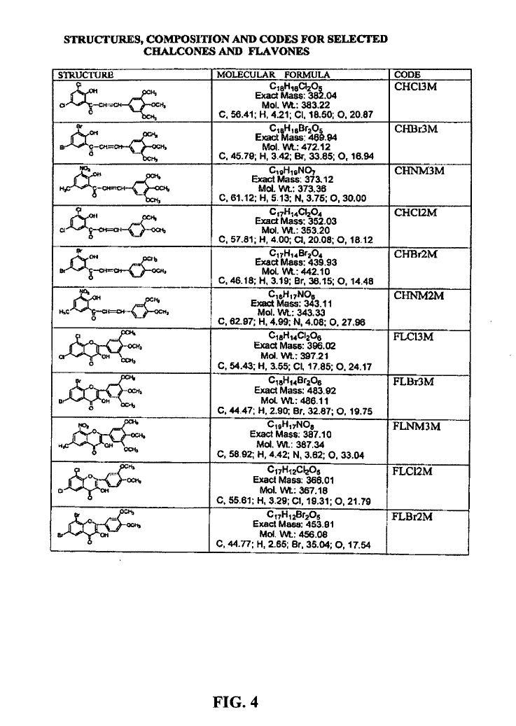

Examining the response of the synthesized compounds to MCF-7 cells in the different drug concentrations reveals that the compounds of the invention, in particular the three (code: CHBr2M, CHCl2M and CHNM3M) show good cytotoxic activities. These three congeners were further evaluated in H9 and PC3 cells. AZT was employed as a positive control in all of these experiments. These data are summarized in FIGS. 1-3. The results show that the compounds of the invention, in particular the two [CHBr2M and CHCI2M] are as good or somewhat better in inhibiting the growth in all the cultures employed.

1. A compound having the structure: 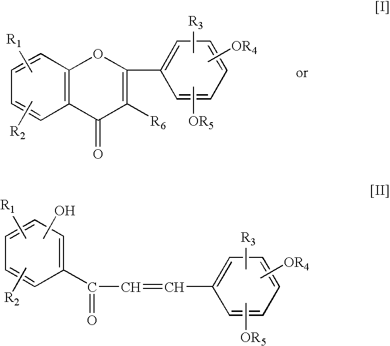
wherein:
R₁ is an electronegative substituent,
R₂ is R₁ or alkyl,
R₃ is H or O-alkyl,
R₄ and R₅ are the same or different and are alkyl and
R₆ is H or OH.

2. A biocompatible compound of claim 1.

3. An anti-cancer compound of claim 1.

4. A compound of claim 3 effective against breast and/or prostate cancer.

5. A compound of claim 1 wherein said electronegative substituent is halogen or NO₂

6. A compound of claim 5 wherein said electronegative substituent is Cl or Br.

7. A compound of claim 1 wherein R₄ and R₅ are each CH₃.

8. A compound of claim 7 having structure [II] wherein R₁ and R₂ are each Cl and R₃ is OCH₃.

9. A compound of claim 7 having structure [II] wherein R₁ and R₂ are each Br and R₃ is OCH₃.

10. A compound of claim 7 having structure [II] wherein R₁ is NO₂, R₂ is CH₃ and R₃ is OCH₃.

11. A compound of claim 7 having structure [II] wherein R₁ and R₂ are each Cl and R₃ is H.

12. A compound of claim 7 having structure [II] wherein R₁ and R₂ are each Br and R₃ is H.

13. A compound of claim 7 having structure [II] wherein R₁ is NO₂, R₂ is CH₃ and R₃ is H.

14. A compound of claim 7 having structure [I] wherein R₆ is OH, R₁ and R₂ are each Cl and R₃ is OCH₃.

15. A compound of claim 7 having structure [I] wherein R₆ is OH, R₁ and R₂ are each Br and R₃ is OCH₃.

16. A compound of claim 7 having structure [I] wherein R₆ is OH, R₁ is NO₂, R₂ is CH₃ and R₃ is OCH₃.

17. A compound of claim 7 having structure [I] wherein R₆ is OH R₁ and R₂ are each Cl and R₃ is H.

18. A compound of claim 7 having structure [I] wherein R₆ is OH R₁ and R₂ are each Br and R₃ is H.

19. A compound of claim 7 having structure [I] wherein R₆ is OH, R₁ is NO₂, R₂ is CH₃ and R₃ is H.

20. A compound of claim 7 having structure [I] wherein R₆ is H; R₁ and R₂ are each Cl and R₃ is OCH₃.

21. A compound of claim 7 having structure [I] wherein R₆ is H; R₁ and R₂ are each Br and R₃ is OCH₃.

22. A compound of claim 7 having structure [I] wherein R₆ is H; R₁ is NO₂, R₂ is CH₃ and R₃ is OCH₃.

22. A compound of claim 7 having structure [I] wherein R₆ is H; R₁ and R₂ are each Cl and R₃ is H.

23. A compound of claim 7 having structure [I] wherein R₆ is H; R₁ and R₂ are each Br and R₃ is H.

24. A compound of claim 7 having structure [I] wherein R₆ is H; R₁ is NO₂, R₂ is CH₃ and R₃ is H.

25. A pharmaceutical composition comprising a therapeutically effective amount of a compound or mixture of compounds of claim 2 and a pharmaceutically acceptable carrier or excipient therefore.

26. A composition of claim 25 suitable for the treatment of cancer.

27. A composition of claim 25 additionally containing at least one biologically active agent different from said compound.

28. A composition of claim 27 suitable for the treatment of cancer.

29. A composition of claim 28 wherein said biologically active agent is an anti-cancer agent.

30. A method for the treatment of a mammal in need thereof comprising administering thereto a therapeutically effective amount of a compound or mixture of compounds of claim 2.

31. A method of claim 30 wherein said mammal is in need of anti-cancer therapy.

32. A method of claim 30 comprising additionally administering to said mammal at least one biologically active agent different from said compound or mixture of compounds.

33. A method of claim 32 wherein said mammal is in need of anti-cancer therapy.

34. A method of claim 33 wherein said biologically active agent is an anti-cancer agent.

35. An article of manufacture comprising packaging material and at least one biologically active agent contained within said packaging material, wherein said at least one therapeutic agent is effective for the treatment of a subject requiring therapy, and wherein said packaging material comprises a label which indicates that said therapeutic agent can be used for at least ameliorating the symptoms with which said subject is afflicted, and wherein said at least one biologically active agent is a compound or mixture of compounds of claim 2.

36. The article of manufacture of claim 35 wherein said subject requires anti-cancer therapy.

37. The article of claim 35 wherein said packaging material additionally contains a biologically active agent different from said compound or mixture of compounds.

38. The article of claim 37 wherein said subject requires anti-cancer therapy.

39. The article of claim 38 wherein said biologically active agent is an anti-cancer agent.

40. A composition for the treatment of a mammal in need thereof in kit comprising, in separate packages: (A) a therapeutically effective amount of a compound or mixture of compounds of claim 2 and (B) a carrier or excipient for said compound or mixture of compounds or a biologically active agent different from said compound or mixture of compounds.

41. A composition of claim 40 suitable for the treatment of cancer.

42. A composition of claim 41 wherein said biologically active agent is an anti-cancer agent.

43. A method for the synthesis of a chalcone of claim 1 by the Claisen condensation of an acetophenone and a benzaldehyde.

44. A method for the synthesis of a flavone of claim 1 by a three step Baker-Venkataraman rearrangement
